(19) United States
(12) Patent Application Publication
(10) Pub. No.: US 2016/0015941 A1
(43) Pub. Date: Jan. 21, 2016

(54) CATHETER ASSEMBLY

(71) Applicants: Terumo Kabushiki Kaisha, Tokyo (JP); Mitsubishi Pencil Company, Limited, Tokyo (JP)

(72) Inventors: Hidenori TANABE, Yamanashi (JP); Yasunobu ZUSHI, Yamanashi (JP)

(73) Assignees: Terumo Kabushiki Kaisha, Tokyo (JP); Mitsubishi Pencil Company, Limited, Tokyo (JP)

(21) Appl. No.: 14/871,082

(22) Filed: Sep. 30, 2015

Related U.S. Application Data
(63) Continuation of application No. PCT/JP2013/059811, filed on Apr. 1, 2013.

(51) Int. Cl. A61M 25/06

(52) U.S. Cl. CPC A61M 25/0618; A61M 25/0606

(57) ABSTRACT

A catheter assembly includes an inner needle having a sharp tip at a distal end thereof; a catheter in which the inner needle is disposed; a tip protective member having an insertion hole through which the inner needle is insertable, the tip protective member being configured to cover at least a tip of the inner needle when the inner needle is withdrawn; and a shutter member which is arranged in an interior of a shutter accommodating section provided in the tip protective member, the shutter member being configured to be deformed and thereby block a movement path of the inner needle in the shutter accommodating section when the inner needle is retracted to a predetermined position relative to the tip protective member. The shutter accommodating section includes a needle guide configured to guide movement of the inner needle.

### CROSS-REFERENCE TO RELATED PATENT APPLICATION

This application is a continuation application of and claims the benefit of priority from International Patent Application No. PCT/JP2013/059811 filed on Apr. 1, 2013, the contents of which are incorporated herein by reference.

### TECHNICAL FIELD

The present disclosure relates to a catheter assembly that can puncture and remain indwelling in a blood vessel when, for example, performing an infusion on a patient.

### BACKGROUND

Conventionally, when an infusion is carried out on a patient, a catheter assembly is used. The catheter assembly is typically equipped with a hollow outer needle (catheter), an outer needle hub (catheter hub) that is fixed to a proximal end of the outer needle, an inner needle that is inserted into the outer needle and has a sharp tip at a distal end thereof, and an inner needle hub that is fixed to a proximal end of the inner needle. In the event that the catheter assembly is used to perform an infusion on a patient, the outer needle, together with the inner needle, punctures a blood vessel of the patient. After puncturing, the inner needle is withdrawn from the outer needle while the outer needle remains in a punctured condition in the patient. Thereafter, a connector, which is provided on a distal end of an infusion tube, is connected to the proximal end of the outer needle hub, and an infusion solution is supplied into the patient's blood vessel through the infusion tube, the outer needle hub, and the outer needle.

During use of this type of catheter assembly, after the inner needle is withdrawn from the outer needle, the inner needle, which has a sharp tip, may be touched inadvertently by the user. In order to prevent this, a catheter assembly has been proposed that is equipped with a protector in which the inner needle can be covered following withdrawal from the outer needle (see, for example, Japanese Laid-Open Patent Publication No. 2002-126080). Further, with the conventional technique, after the tip of the inner needle has been covered by the protector, in order to prevent the inner needle from protruding from the distal end of the protector when advancing the inner needle in the distal end direction (reverting), a configuration has also been adopted in which a shutter member is arranged within the protector for blocking the movement path of the inner needle accompanying retraction of the inner needle.

### SUMMARY

For the shutter member, there may be used, for example, a V-shaped metallic member, which is formed by bending a middle portion of a plate-shaped metallic member into a V-shape. The protector includes a shutter accommodating section for housing the shutter member such that a V-shaped apex of the shutter member faces a distal end side of the protector. In the protector, on both a distal end side and a proximal end side of the shutter accommodating section, lumens (hereinafter referred to respectively as a “distal end side lumen” and a “proximal end side lumen”) are formed for enabling insertion of the inner needle.

During an assembly process in which the inner needle is inserted into the protector, the inner needle is inserted through the distal end side lumen from a distal end opening in the protector in a state in which the shutter is housed in the shutter accommodating section. The inner needle is then moved in the proximal end direction relative to the protector. At that time, it is necessary for the inner needle to elastically compress and deform the shutter member in order to be inserted into the proximal end side lumen in the protector.

However, when the inner needle is inserted into the protector, if the distal end of the shutter member (the V-shaped apex) is positioned in front of the distal end side lumen in the protector, the proximal end of the inner needle may become engaged with or caught on the distal end of the shutter member. Thus, cases may occur in which it becomes difficult for the inner needle to be inserted beyond this position in the direction of the proximal end.

Further, when the proximal end of the inner needle passes through the shutter accommodating section in the proximal end direction while elastically compressing and deforming the shutter member, the inner needle receives a reaction force from the side thereof due to the elastic force of the shutter member. In this case, depending on the rigidity of the inner needle and the elastic force of the shutter member, the proximal end portion of the inner needle may be bent laterally within the shutter accommodating section, thereby making it difficult for the proximal end of the inner needle to be inserted into the proximal end side lumen in the protector.

Certain embodiments of the present invention has been devised taking into consideration the aforementioned difficulties, and has an object of certain embodiments of providing a catheter assembly which, during an assembly process, enables the inner needle to be easily inserted into a lumen of the protector in a state in which the shutter member is accommodated in the protector.

A catheter assembly according to an embodiment of the present invention includes an inner needle having a sharp tip at a distal end thereof, a catheter through which the inner needle is inserted, and a tip protective member having an insertion hole through which the inner needle can be inserted. The tip protective member is configured to cover at least the tip of the inner needle when the inner needle is withdrawn. The catheter assembly further includes a shutter member, which is arranged in the interior of a shutter accommodating section provided in the tip protective member. The shutter member is configured to be deformed and thereby block a movement path of the inner needle in the shutter accommodating section when the inner needle is retracted to a predetermined position relative to the tip protective member. The shutter accommodating section includes a needle guide alongside the movement path of the inner needle, the needle guide being configured to guide movement of the inner needle.

According to the above configuration, since the needle guide is provided in the shutter accommodating section, the distal end portion of the shutter member can be easily positioned to one side (a side opposite to the guide member) in the interior of the shutter accommodating section. Therefore, during the assembly process, when the inner needle is inserted from the distal end of the tip protective member, it is difficult for the proximal end of the inner needle to become caught on the distal end of the shutter member. Furthermore, during the assembly process, when the proximal end of the inner needle is made to move in the proximal end direction in the interior of the shutter accommodating section while the shutter member is elastically compressed and deformed, although the inner needle receives a reaction force from the shutter member, because the inner needle is supported laterally by the needle guide, deformation (deflection) of the inner needle as a result of the reaction force from the shutter member is suppressed. Thus, the proximal end of the inner needle can be inserted smoothly into the insertion hole of the proximal end side of the shutter accommodating section. Consequently, with the catheter assembly according to certain embodiments of the present invention, during the assembly process, the inner needle can be easily inserted into the lumen of the tip protective member in a state in which the shutter member is accommodated therein.

In the aforementioned catheter assembly, a distal end portion of the shutter member may be disposed between the needle guide and an inside wall that faces the needle guide in the shutter accommodating section.

With the above configuration, the distal end portion of the shutter member can be effectively shifted toward one side of the shutter accommodating section and, during the assembly process in which the inner needle is inserted into the inner tube, engagement of the proximal end of the inner needle with the distal end portion of the shutter member can be more effectively suppressed.

In the aforementioned catheter assembly, the shutter member may be formed in a V-shape, and an apex of the V-shape may constitute the distal end portion of the shutter member.

According to this arrangement, the shutter member formed in a V-shape can be disposed appropriately and ease of assembly is enhanced.

In the catheter assembly, the insertion hole may have an accommodating space in the interior of the shutter accommodating section, a distal end side lumen may extend from a distal end of the accommodating space, and a proximal end side lumen may extend from a proximal end of the accommodating space. In addition, the needle guide may have a guide surface that continues from a proximal end of the distal end side lumen.

According to the above structure, during the assembly process, upon insertion of the inner needle from the distal end of the tip protective member, because the inner needle, which projects out from the distal end side lumen, is guided effectively by the guide surface, the inner needle, which receives the reaction force from the shutter member, is supported effectively and the proximal end of the inner needle can be guided more smoothly into the proximal end side lumen.

In the aforementioned catheter assembly, the distal end portion of the proximal end side lumen may include a tapered inner surface with an inner diameter that expands outwardly in the direction of the distal end. A radius of a distal end opening in the tapered inner surface of the proximal end side lumen may be greater than a distance between the guide surface and a common center line shared by the distal end side lumen and the proximal end side lumen.

According to the above structure, when the inner needle is inserted from the distal end of the tip protective member and even in the case that the inner needle undergoes a deflection upon receiving the reaction force from the shutter member, the tapered inner surface of the proximal end side lumen, which opens widely and outwardly relative to the position of the guide surface, allows the proximal end of the inner needle to be inserted smoothly into the proximal end side lumen.

In the catheter assembly, the distal end portion of the proximal end side lumen may include a tapered inner surface with an inner diameter that expands outwardly in the direction of the distal end. In addition, a distance from the distal end of the distal end side lumen to a proximal end of the guide surface may be greater than a distance from the proximal end of the guide surface to a proximal end of the tapered inner surface. According to this structure, by setting in an elongated manner a portion (the distal end side lumen and the guide surface) that supports the reaction force that the inner needle receives from the shutter member, deflection of the inner needle by the reaction force received from the shutter member can be suitably suppressed, and the proximal end of the inner needle can be inserted more smoothly into the proximal end side lumen.

In the aforementioned catheter assembly, the distal end portion of the proximal end side lumen may include a tapered inner surface with an inner diameter that expands outwardly in the direction of the distal end. A diameter of the proximal end of the distal end side lumen may be less than a diameter of a portion of the proximal end side lumen that is disposed more proximally than the tapered inner surface. According to such a structure, the proximal end of the inner needle can be inserted more smoothly into the proximal end side lumen.

### BRIEF DESCRIPTION OF THE DRAWINGS

FIG. 1 is a perspective view showing an overall structure of a catheter assembly according to an embodiment of the present invention.

FIG. 2 is a vertical cross-sectional partial view of the catheter assembly shown in FIG. 1.

FIG. 3A is a vertical cross-sectional partial view of the catheter assembly shown in FIG. 1 taken along a line IIIA-IIIA of FIG. 2.

FIG. 3B is a vertical cross-sectional partial view of the catheter assembly shown in FIG. 1 showing a condition in which a catheter member and a protector are separated from each other.

FIG. 4 is a perspective view of an inner tube in which a shutter member is accommodated.

FIG. 5 is a perspective view of the shutter member and an inner tube forming part.

FIG. 6 is a perspective view as observed from a distal end side of a distal end portion of a first part.

FIG. 7 is a perspective view as observed from a proximal end side of the distal end portion of the first part.

FIG. 8 is a plan view of the distal end portion of the first part.

FIG. 9 is a vertical cross-sectional view showing a condition in which the catheter hub and the protector are separated from one another and a tip of an inner needle is covered by the protector.

FIGS. 10A-10C are cross-sectional views showing a process of inserting an inner needle from a distal end side of an inner tube in which the shutter member is accommodated.

### DETAILED DESCRIPTION OF THE EMBODIMENTS

Hereinafter, embodiments of a catheter assembly according to the present invention will be described in detail below with reference to the accompanying drawings.

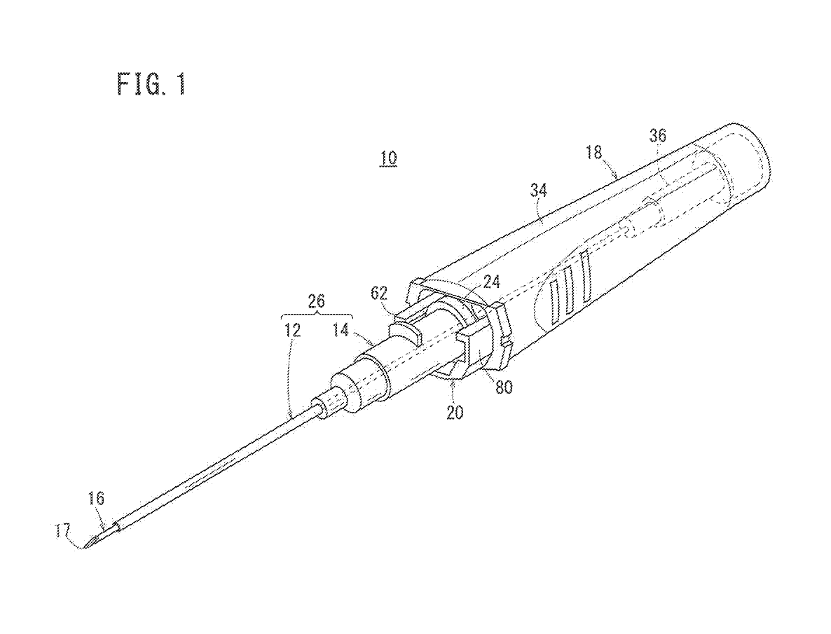

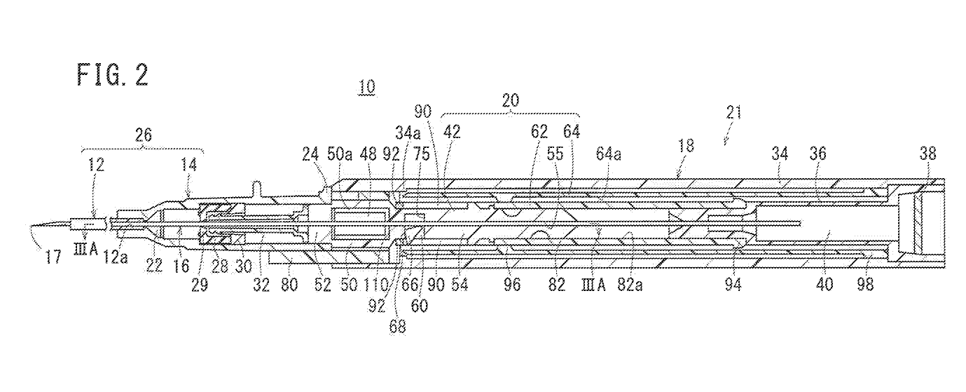

FIG. 1 is a perspective view showing the overall structure of a catheter assembly 10 according to an embodiment of the present invention. FIG. 2 is a vertical cross-sectional partial view of the catheter assembly 10.

As shown in FIG. 1, the catheter assembly 10 is equipped with a tubular catheter 12 that functions as an outer needle, a catheter hub 14 that is connected to a proximal end side of the catheter 12, a tubular inner needle 16 having a sharp tip 17 on a distal end thereof and which is capable of being inserted through the interior of the catheter 12, an inner needle hub 18 connected to a proximal end side of the inner needle 16, and a protector 20 that covers the tip 17 of the inner needle 16 when the inner needle 16 is retracted. The catheter assembly 10 may be used in the following manner, described in detail below.

A user (e.g., a doctor or nurse) performs an operation by gripping the inner needle hub 18 of the catheter assembly 10, whereby the distal end portion thereof punctures and is inserted into a blood vessel of a patient. In an initial condition, prior to use of the catheter assembly 10 (i.e., before puncturing the patient), the inner needle 16 is inserted through the catheter 12 to form a double tube structure in which the inner needle 16 projects a predetermined length from the distal end of the catheter 12. Further, in the initial condition of the catheter assembly 10, the proximal end side of the catheter hub 14 and the distal end side of the inner needle hub 18 are connected through the protector 20. Hereinafter, the initial condition of the catheter assembly 10 will be referred to as a “puncture enabled state.”

In the puncture enabled state of the catheter assembly 10, the catheter 12 and the inner needle 16 that make up the double tube structure are inserted together into the blood vessel of the patient. After puncturing the patient, in a condition in which the position of the catheter 12 is maintained, the inner needle hub 18 is retracted in the direction of the proximal end, whereby the protector 20 is made to separate away from the catheter hub 14. At the same time, the inner needle 16, which is connected to the inner needle hub 18, is withdrawn integrally therewith, and the inner needle 16 and the inner needle hub 18 are detached from the catheter 12 and the catheter hub 14 (hereinafter, a coupling body made up from the catheter 12 and the catheter hub 14 will be referred to as a “catheter member 26”). As a result, in the catheter assembly 10, a state is brought about in which only the catheter member 26 is left indwelling on the side of the patient.

When the inner needle 16 is withdrawn from the catheter 12, the inner needle 16 becomes accommodated inside the protector 20 due to the protector 20 extending in the distal end direction with respect to the inner needle hub 18. Consequently, exposure of the inner needle 16 to the exterior is prevented. After the inner needle 16 has been withdrawn from the catheter 12, a non-illustrated connector of an infusion tube is connected to the proximal end side of the catheter hub 14, whereby an infusion agent (medicinal solution) may be supplied from the infusion tube to the patient.

Next, the structure of the catheter assembly 10 will be described in greater detail.

In the puncture enabled state, the catheter assembly 10 is constituted as a single assembly, in which the double tube structure of the catheter 12 and the inner needle 16, the catheter hub 14, the protector 20, and the inner needle hub 18 are combined and are capable of being handled integrally.

The catheter 12, which is constituted as an outer needle in the catheter assembly 10, is a flexible and narrow diameter tubular member formed with a predetermined length. As shown in FIG. 2, in the interior of the catheter 12, a lumen 12a is formed to extend and penetrate through the catheter 12 in the axial direction. The inner diameter of the lumen 12a is set to a size that enables the inner needle 16 to be inserted through the lumen 12a.

The catheter 12 may be made from a resin material, in particular, a soft resin material. For example, a fluororesin, such as polytetrafluoroethylene (PTFE), ethylene-tetrafluoroethylene copolymer (ETFE), perfluoroalkoxy fluororesin (PFA), etc., an olefin resin, such as polyethylene, polypropylene, etc., or a mixture thereof, polyurethane, polyester, polyamide, polyether nylon resin, and a mixture of the olefin resin and ethylene-vinyl acetate copolymer may be used. In addition, the catheter 12 may be made from a transparent resin material, so that all or a portion of the interior is visible.

The catheter hub 14 is connected in a fixed manner to the proximal end of the catheter 12. The distal end portion of the catheter hub 14 and the proximal end portion of the catheter 12 are fixed together in a liquid-tight manner by a crimp pin 22 (see FIG. 2), which is arranged on an inner side of the distal end portion of the catheter hub 14.

When the catheter assembly 10 is used, the catheter hub 14 is exposed on the patient's skin in a state in which the catheter 12 has pierced into the blood vessel, and is pasted and held in place on the skin by tape or the like. The catheter hub 14 preferably is made from a material that is more rigid than the catheter 12. The constituent material of the catheter hub 14 is not limited to any particular material. However, a thermoplastic resin material, such as polypropylene, polycarbonate, polyamide, polysulfone, polyarylate, methacrylate-butylene-styrene copolymer, etc., may be used.

As shown in FIG. 2, in the present embodiment, a hemostasis valve 28, a seal member 30, and a plug 32 are arranged in the interior of the catheter hub 14.

The hemostasis valve 28 includes a valve member 29 formed with a slit 90 in a distal end thereof such that when blood flows into the catheter hub 14 through the lumen 12a of the catheter 12 after puncturing of the blood vessel by the catheter 12, flow of blood to the proximal end side of the catheter hub 14 is prevented. The seal member 30, which is made from a material (e.g., a porous body) that allows for the flow of gas yet blocks the flow of liquid through the seal member 30, is arranged on the proximal end side of the hemostasis valve 28.

The plug 32 is formed in a tubular shape and is arranged movably in the axial direction in the interior of the catheter hub 14. When the catheter hub 14 and the connector of the infusion tube are connected, and the plug 32 is then moved by the connector in the direction of the distal end, the plug 32 penetrates through the hemostasis valve 28. As a result, a condition is brought about in which the infusion solution can be supplied from the infusion line to the blood vessel through the catheter member 26 (the catheter hub 14 and the catheter 12).

The inner needle 16 is a rigid tubular member that is capable of puncturing the patient's skin. The inner needle is sufficiently longer than the catheter 12 such that, in the puncture enabled state (initial condition) of the catheter assembly 10, the tip 17 of the inner needle 16 projects from a distal end opening of the catheter 12. Further, in the puncture enabled state, an intermediate location in the longitudinal direction of the inner needle 16 is inserted through the interior of the catheter hub 14 and the proximal end side thereof is retained inside the inner needle hub 18. As a constituent material of the inner needle 16, a metal material, such as stainless steel, aluminum or aluminum alloy, titanium or titanium alloy, may be used.

The inner needle hub 18 is equipped with a hollow hub main body 34, and a hollow inner needle retaining member 36 that is fitted into the proximal end side of the hub main body 34. The hub main body 34 is formed with an appropriate size (thickness, length) to enable the hub main body 34 to be gripped and operated easily by the user when using the catheter assembly 10. The inner needle retaining member 36 is fitted into and fixed to the proximal end side of the hub main body 34 and fixedly retains the proximal end portion of the inner needle 16. The distal end portion of the inner needle retaining member 36 serves to retain (tightly fix) the proximal end side of the inner needle 16, and the proximal end portion of the inner needle retaining member 36 is fitted onto an inner surface of the hub main body 34.

A filter 38, which blocks the flow of liquid yet allows air to pass therethrough, is arranged inside the proximal end portion of the inner needle retaining member 36. When the inner needle 16 and the catheter 12 puncture the patient, blood flows through the inner needle 16 into a flashback chamber 40 that is formed in the interior of the inner needle retaining member 36. By blood flowing into the flashback chamber 40, the user can determine whether or not the inner needle 16 and the catheter 12 have punctured the patient normally.

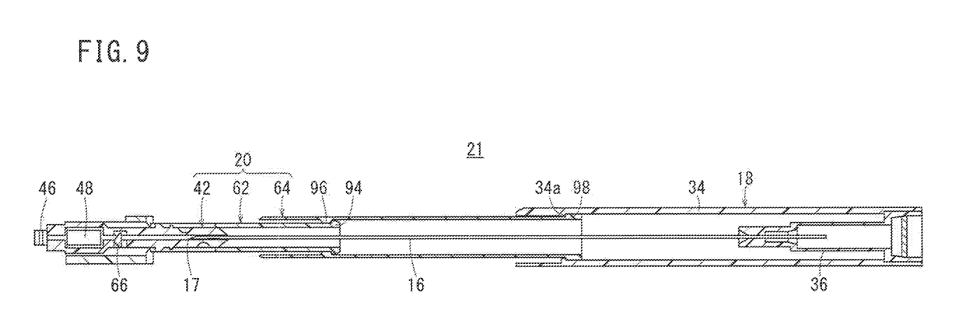

When the inner needle 16 is withdrawn from the catheter 12, the protector 20 covers the tip 17 of the inner needle 16 upon the inner needle 16 being accommodated in the protector 20. As shown in FIG. 2, the protector 20 includes an inner tube 42 that releasably engages with the proximal end of the catheter hub 14 and an outer tube 62 having the inner tube 42 arranged inside thereof and which is capable of being displaced relatively with respect to the inner tube 42 within a restricted range in the axial direction. The protector 20 further includes a shutter member 48 disposed in the interior of the inner tube 42 and a joint tube 64 that is inserted inside the outer tube 62 and is capable of sliding axially with respect to the outer tube 62. When a withdrawing operation of the inner needle 16 from the catheter 12 is carried out, the protector 20 is extended to cover the total length of the inner needle 16 (see FIG. 9).

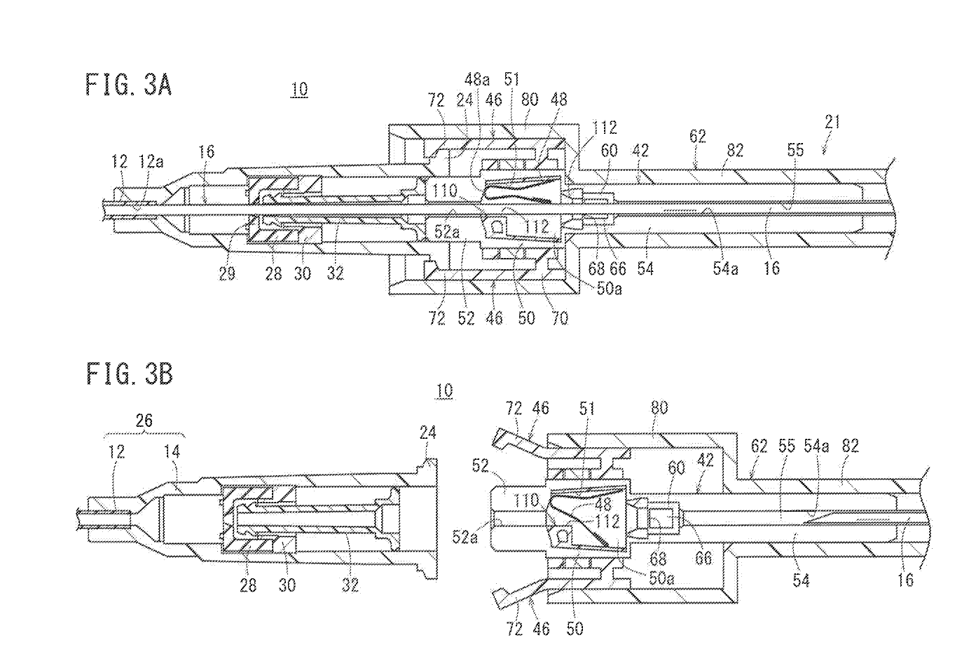

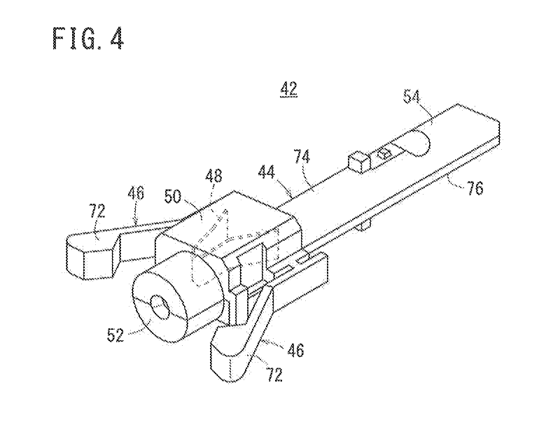

The inner tube 42 functions as a tip protective member, which covers the tip 17 of the inner needle 16 accompanying withdrawal of the inner needle 16 from the catheter 12. FIG. 3A is a vertical cross-sectional partial view taken along the line IIIA-IIIA of FIG. 2, and FIG. 3B is a vertical cross-sectional partial view showing a condition in which the catheter member 26 and the protector 20 are separated from each other. In FIGS. 3A and 3B, the inner needle hub 18 and the joint tube 64 are omitted from illustration. FIG. 4 is a perspective view of the inner tube 42 in which the shutter member 48 is accommodated.

As shown in FIGS. 3A to 4, the inner tube 42 includes a hollow inner tube main body 44 and arms 46 that are disposed integrally on the outer side of the inner tube main body 44. The inner tube main body 44 includes a shutter accommodating section 50 in which the shutter member 48 is accommodated, a distal end tubular section 52 that projects from and is contiguous to a distal end side of the shutter accommodating section 50, and a proximal end tubular section 54 that projects from and is contiguous to the proximal end side of the shutter accommodating section 50. Further, an insertion hole 55, which penetrates through the distal end tubular section 52, the shutter accommodating section 50, and the proximal end tubular section 54 in the axial direction, and through which the inner needle 16 can be inserted, is disposed in the inner tube main body 44.

When the inner needle 16 is retracted to a predetermined position relative to the inner tube 42, the shutter member 48 is deformed to thereby block the movement path of the inner needle 16 in the shutter accommodating section 50. In the illustrated example, the shutter member 48 is an elastic member, which is formed by bending a plate-shaped member into a V-shape. In a condition in which the V-shaped apex of the shutter member 48 is oriented toward the distal end side of the inner tube 42, the shutter member 48 is disposed in an accommodating space 50a, which is formed inside the shutter accommodating section 50. The accommodating space 50a makes up a portion of the insertion hole 55 through which the inner needle 16 can be inserted.

In an initial state of the catheter assembly 10 shown in FIG. 3A, the inner needle 16 penetrates back and forth through the accommodating space 50a. In this state, the shutter member 48 is elastically compressed and deformed due to being pressed by a side surface of the inner needle 16 such that the shutter member 48 is placed in a state of being closed into a smaller shape. As a constituent material of the shutter member 48, a pseudoelastic alloy (including a superelastic alloy), such as a Ni—Ti alloy, a shape memory alloy, stainless steel, a cobalt-based alloy, noble metals, such as gold and platinum, a tungsten-based alloy, and carbon-based materials, etc., may be used.

In the initial state of the catheter assembly 10 shown in FIG. 3A, the distal end tubular section 52 is fitted into the proximal end of the catheter hub 14. A lumen 52a of the distal end tubular section 52 (hereinafter referred to as a “distal end side lumen 52a”) is a hollow portion that extends in the direction of the distal end from the accommodating space 50a in the interior of the shutter accommodating section 50. The distal end side lumen 52a makes up another portion of the insertion hole 55 through which the inner needle 16 can be inserted.

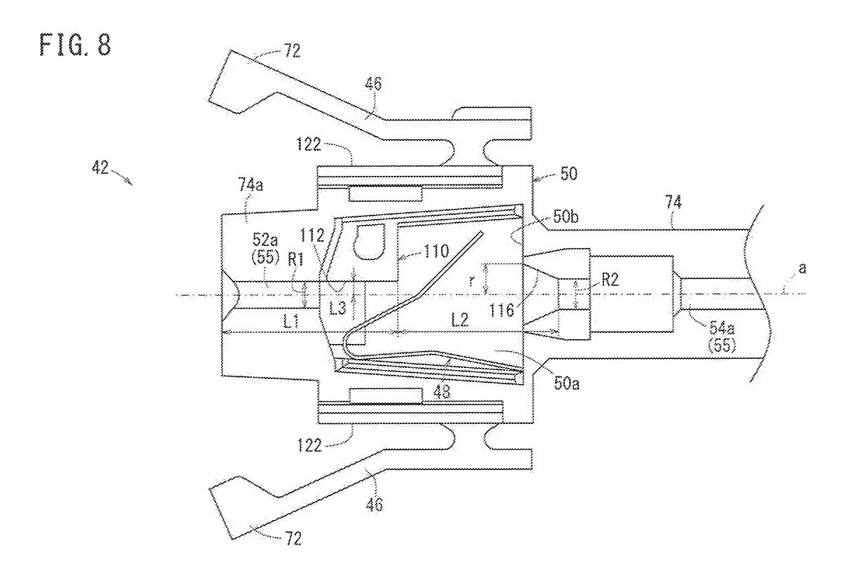

The proximal end tubular section 54 is a tubular body, which is longer in comparison to the distal end tubular section 52. A lumen 54a of the proximal end tubular section 54 (hereinafter referred to as a “proximal end side lumen 54a”) is a hollow portion that extends in the direction of the proximal end from the accommodating space 50a in the interior of the shutter accommodating section 50. The proximal end side lumen 54a makes up another portion of the insertion hole 55 through which the inner needle 16 can be inserted. The distal end side lumen 52a and the proximal end side lumen 54a possess a common center line a (see FIG. 8) and communicate with one another through the accommodating space 50a.

As shown in FIGS. 2 and 3A, at a location near the distal end of the proximal end tubular section 54, a side hole 60 is provided that communicates between the interior and the exterior of the proximal end tubular section 54. A stopper 66, which engages releasably with respect to the outer tube 62, is disposed in the side hole 60. The stopper 66 is formed integrally with the proximal end tubular section 54 through an elastically deformable hinge 68 that is interposed between the stopper 66 and the proximal end tubular section 54.

The arms 46 provided on the inner tube 42 are capable of releasably engaging, from the outside, with the proximal end of the catheter hub 14. In the illustrated example, the arms 46 are provided as a pair located on left and right side surfaces of the shutter accommodating section 50. In a condition in which engaging end sections 72, which are disposed on distal end sides of the respective arms 46, are subjected to no external force and thus are in a free state, the engaging end sections 72 are tilted and expanded outwardly in the direction of the distal end. By elastically deforming intermediate locations in the longitudinal direction of the respective arms 46, the engaging end sections 72 are capable of being displaced perpendicularly (in the illustrated example, laterally of the inner tube 42) with respect to the axial direction of the inner tube main body 44.

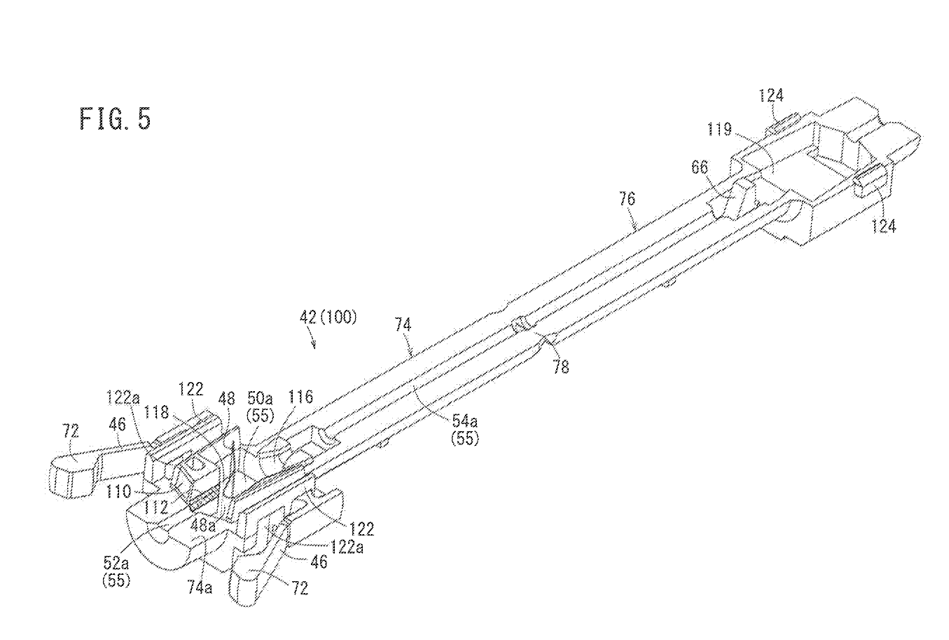

In the present embodiment, the inner tube 42 is formed by bending an inner tube forming part 100, shown in FIG. 5, in half at an intermediate position in the longitudinal direction thereof. More specifically, the inner tube 42 is constituted from a first part 74 that makes up one side portion (an upper portion shown in FIG. 4), and a second part 76 that makes up another side portion (a lower portion shown in FIG. 4) with reference to the axis (lumen) thereof. The inner tube 42 in the illustrated example is an integrally molded piece in which the respective proximal ends of the first part 74 and the second part 76 are connected by a hinge 78. The insertion hole 55 is formed between the first part 74 and the second part 76 as a result of the first part 74 and the second part 76 being overlapped in a predetermined positional relationship.

As shown in FIG. 5, a first installation groove 118 in which a portion of the shutter member 48 is arranged is provided on the first part 74. A second installation groove 119 in which another portion of the shutter member 48 is arranged is provided on the second part 76.

In the illustrated example, the first part 74 mainly constitutes the upper side of the inner tube 42, and the second part 76 mainly constitutes the lower side of the inner tube 42. The aforementioned pair of arms 46 is disposed integrally at the first part 74, and the stopper 66 is disposed at the second part 76. The pair of arms 46 may also be provided on the second part 76 that makes up the lower side part. The stopper 66 may also be provided on the first part 74 that makes up the upper side part.

The shutter member 48 is disposed inside the shutter accommodating section 50 (in the shutter accommodating space 50a) so as to be elastically deformable in an intersecting direction with respect to the overlapping direction of the first part 74 and the second part 76 (perpendicularly with respect to the overlapping direction in the illustrated example). Therefore, the shutter member 48 does not press the first part 74 and the second part 76 in a direction that mutually separates the first part 74 and the second part 76 away from each other. Further, even in the case that the inner needle 16 returns back in the distal end direction after the tip 17 of the inner needle 16 has been accommodated in the inner tube 42, whereby the shutter member 48 is pressed by the inner needle 16, the inner tube 42 is not split off thereby. Further, a distal end portion 48a (V-shaped apex) of the shutter member 48 is oriented towards the distal end side of the inner tube 42.

On the first part 74, a pair of engagement pieces 122 having openings 122a therein are provided on outer sides of the first installation groove 118. On the second part 76, a pair of engaging claws 124, which engage with the engagement pieces 122 through the openings 122a thereof, are provided on outer sides of the second installation groove 119. Through engagement of the engagement pieces 122 and the engaging claws 124, the first part 74 and the second part 76 are retained and held in an overlapped state (a state in which the inner tube 42 is closed). Alternatively, the engaging claws 124 may be provided on the first part 74, and the engagement pieces 122 may be provided on the second part 76.

With the catheter assembly 10 according to certain embodiments of the present invention, as shown in FIG. 3A, the shutter accommodating section 50 includes a needle guide 110 for guiding movement of the inner needle 16 on the side of the movement path of the inner needle 16. In the illustrated example, the needle guide 110 is disposed so as to constitute a stepped portion in the interior of the shutter accommodating section 50 and is positioned on the distal end side and to one of the left and right sides of the shutter accommodating section 50 interior (accommodating space 50a).

As shown in FIG. 5, the needle guide 110 is disposed integrally on the first part 74 and projects outwardly beyond a divided surface 74a of the first part 74 at which the first part 74 and the second part 76 are split off. More specifically, a portion (roughly one half) of the needle guide 110 is disposed in the first installation groove 118, and the remaining portion (the remaining one half) of the needle guide 110 is inserted into the second installation groove 119. Alternatively, one portion (roughly one half) of the needle guide 110 may be disposed in the first installation groove 118 and the other portion (the remaining one half) of the needle guide 110 may be disposed in the second installation groove 119 such that, in a state in which the inner tube 42 is formed by overlapping the first part 74 and the second part 76, the needle guide 110 is constituted by the one portion and the remaining portion.

The needle guide 110 is provided with a guide surface 112 (the portion shown in cross hatching in FIG. 5) that continues from the proximal end of the distal end side lumen 52a. In the illustrated example, the guide surface 112 is substantially flat. However, the guide surface may be provided as a groove that is arcuately shaped in cross section and extends along the axial direction of the inner tube 42. As shown in FIG. 3A, the distal end portion 48a (the apex of the V-shape) of the shutter member 48 is disposed between the needle guide 110 and an inside wall 51 of the shutter accommodating section 50 that faces the needle guide 110.

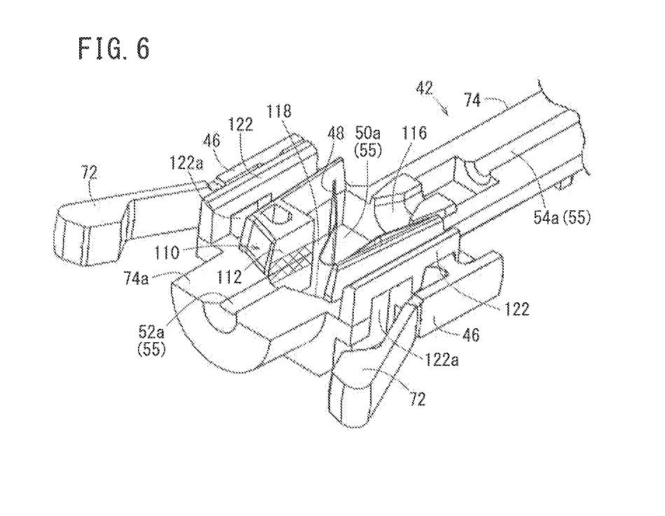

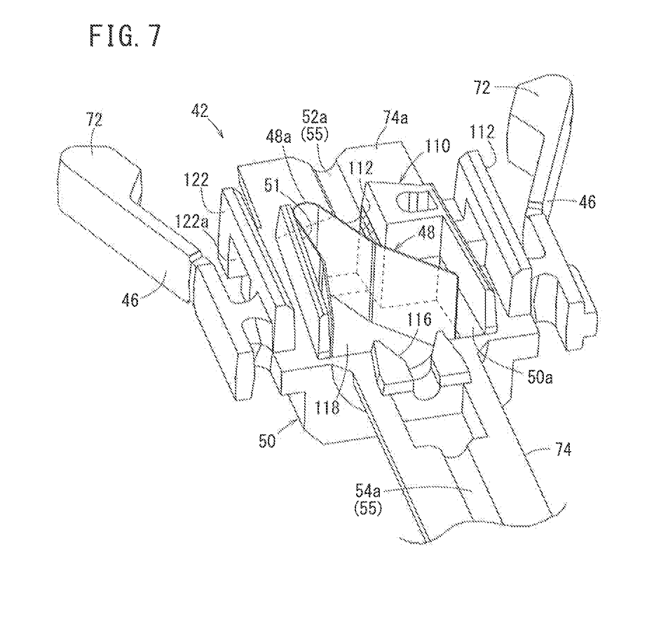

As shown in FIGS. 6 to 8, the distal end portion of the proximal end side lumen 54a includes a tapered inner surface 116 with an inner diameter that expands outwardly in the direction of the distal end. As shown in FIG. 8, the distance L1 from the distal end of the distal end side lumen 52a to a proximal end of the guide surface 112 is greater than the distance L2 from the proximal end of the guide surface 112 to the proximal end of the tapered inner surface 116. The diameter R1 of the proximal end of the distal end side lumen 52a is less than the diameter R2 of a portion of the proximal end side lumen 54a that is disposed more proximally than the tapered inner surface 116. The radius r of the distal end opening in the tapered inner surface 116 of the proximal end side lumen 54a is greater than the distance L3 between the guide surface 112 and a common center line a that is shared by the distal end side lumen 52a and the proximal end side lumen 54a.

Next, the structure of the outer tube 62 will be described. As shown in FIG. 3A, the outer tube 62 includes an arm accommodating section 80 in which the arms 46 can be accommodated, and a tubular section 82 that projects from the proximal end side of the arm accommodating section 80. The outer tube 62 corresponds to an outside member, which is arranged on an outer side of the inner tube 42 that functions as the tip protective member.

The arm accommodating section 80 is formed in a box-shape and is opened on an upper portion and an distal end portion thereof (see FIG. 1). In the puncture enabled state of the catheter assembly 10, the proximal end of the catheter hub 14 and the distal end side of the inner tube 42 (the pair of arms 46 and the shutter accommodating section 50) are arranged in the interior of the arm accommodating section 80.

As shown in FIG. 2, in the tubular section 82 of the outer tube 62, a lumen 82a that communicates with the interior of the arm accommodating section 80 is formed to penetrate in the axial direction. On upper and lower portions at locations near the distal end of the tubular section 82, a pair of two elongate slits 90, which penetrate through the inside and outside of the tubular section 82, are disposed along the axial direction of the tubular section 82.

At the distal end of the tubular section 82, a pair of two engagement members 92 are provided that correspond to the pair of slits 90. The two engagement members 92 are disposed to project toward the inside of an opening that is provided on the proximal end of the arm accommodating section 80. The engagement members 92 are capable of being elastically deformed outwardly when pressing forces are applied thereto from the inside. An outer side hook 94, which projects outwardly and extends in a circumferential direction, is provided on the outer side surface of the proximal end of the outer tube 62.

As shown in FIG. 2, the joint tube 64 includes a lumen 64a in which the tubular section 82 of the outer tube 62 can be accommodated, and is assembled in a slidable manner relative to the outer tube 62. An inner side hook 96, which projects inwardly and extends in a circumferential direction, is provided on the inner side surface of a portion of the joint tube 64 that is positioned near the distal end. The inner side hook 96 is capable of engaging with the outer side hook 94 that is provided on the outer tube 62. An outer side hook 98, which projects outwardly and extends in a circumferential direction, is provided on the outer side surface of the proximal end of the joint tube 64. The outer side hook 98 is capable of engaging with a step 34a that is provided on the inner circumferential portion of the distal end side of the hub main body 34.

The materials that may constitute the aforementioned members (the hub main body 34, the inner needle retaining member 36, the inner tube 42, the outer tube 62, the joint tube 64) of the inner needle hub 18 and the protector 20 are not particularly limited and may be, for example, the same materials as given in the description of the catheter hub 14. In this case, all of these members may be formed from the same material or may be formed from different materials for each of the members.

As shown in FIG. 2, in the initial condition (puncture enabled state) of the catheter assembly 10, the inner needle 16 is inserted into the catheter 12, and the tip 17 projects a predetermined length from the distal end of the catheter 12. In addition, the distal end tubular section 52 of the inner tube 42 is inserted into the proximal end of the catheter hub 14, and the outer tube 62 is moved maximally within the movable range thereof toward the distal end side with respect to the inner tube 42. Further, as shown in FIG. 3A, the pair of arms 46 provided on the inner tube 42 are positioned inside the arm accommodating section 80 of the outer tube 62, whereby the arms 46 are placed in a closed state. The closed pair of arms 46 engages with a flange 24 of the catheter hub 14, whereby separation of the catheter hub 14 from the protector 20, including the inner tube 42, is prevented.

Further, as shown in FIG. 2, in the initial condition (puncture enabled state) of the catheter assembly 10, the tip 17 of the inner needle 16 is positioned more on the distal end side than the stopper 66. The stopper 66, which is pressed against the outer side surface of the inner needle 16, projects outwardly from the proximal end tubular section 54 of the inner tube 42 and, by engagement with the engagement members 92 through the lower side slit 90 that is provided on the outer tube 62, movement of the outer tube 62 in the proximal end direction with respect to the inner tube 42 is prevented. Furthermore, the joint tube 64 is inserted maximally into the inner needle hub 18 and the tubular section 82 of the outer tube 62 is inserted maximally into the joint tube 64. In this state, the arm accommodating section 80 is inserted into the distal end side of the inner needle hub 18. Moreover, the shutter member 48 is held in an elastically deformed state by the inner needle 16 nearer to one side of the accommodating space 50a.

In the puncture enabled state, a user (a doctor or nurse, etc.) performs an operation by gripping the inner needle hub 18 of the catheter assembly 10, whereby the catheter 12 and the inner needle 16 thereof can puncture and be inserted into a blood vessel of a patient. After puncturing, a detachment operation (withdrawing operation of the inner needle 16) is carried out to detach the coupling body (referred to below as an “inner needle unit 21”), which is made up from the inner needle 16, the inner needle hub 18, and the protector 20, from the catheter member 26.

During the detachment operation, in a state in which the position of the catheter member 26 is maintained, the inner needle hub 18 is retracted in the proximal end direction. Upon doing so, the inner needle 16, which is retained by the inner needle retaining member 36 of the inner needle hub 18, starts to be retracted with respect to the catheter 12. At the same time, a condition (a movement stopped state) is maintained where the protector 20 cannot be displaced with respect to the catheter member 26, until the inner needle 16 has been retracted by a predetermined amount.

When the inner needle hub 18 is retracted by a predetermined amount, the step 34a (see FIG. 2), which is disposed on the distal end side of the hub main body 34, engages with the outer side hook 98 located on the proximal end side of the joint tube 64. Therefore, accompanying the retraction of the hub main body 34, the joint tube 64 also is retracted. Upon further retraction of the hub main body 34, the inner side hook 96 on the distal end side of the joint tube 64 engages with the outer side hook 94 on the proximal end side of the tubular section 82. Thus, in this state, a condition is brought about in which the outer tube 62, the joint tube 64, and the inner needle hub 18 are expanded to the maximum extent. Further, in this state, together with the retraction of the inner needle hub 18 with respect to the joint tube 64 and the retraction of the joint tube 64 with respect to the outer tube 62, the protector 20 also is expanded. As shown in FIG. 9, the inner needle 16 is then covered over its entire length by the inner needle hub 18 and the protector 20.

On the other hand, during the process of retracting the inner needle hub 18 with respect to the catheter member 26, the inner needle 16 also is retracted with respect to the inner tube 42. At this time, when the tip 17 (see FIG. 2) of the inner needle 16 moves more toward the proximal end side than the shutter member 48 that is arranged inside the inner tube 42, the shutter member 48 expands into the accommodating space 50a due to the elastic restoring force thereof. As a result, since the passage in which the inner needle 16 can move through the accommodating space 50a is blocked, the tip 17 of the inner needle 16 is prevented from projecting out again from the distal end of the inner tube 42. Nonetheless, at this time, the arms 46 provided on the inner tube 42 remain closed, and engagement between the inner tube 42 and the catheter hub 14 is maintained.

When the inner needle 16 is further retracted within the inner tube 42, and the tip 17 thereof moves more toward the proximal end side than the stopper 66 that is disposed in the inner tube 42, under the elastic restoring force of the hinge 68, the stopper 66 is displaced inwardly of the inner tube 42. Consequently, since the engagement between the stopper 66 and the engagement members 92 disposed on the outer tube 62 is released, the outer tube 62 becomes capable of being displaced in the proximal end direction with respect to the inner tube 42. Therefore, from the condition in which the outer tube 62, the joint tube 64, and the inner needle hub 18 are displaced relatively and expanded maximally in the axial direction, when a movement operation is performed to move the inner needle hub 18 further in the proximal end direction, the outer tube 62 is displaced in the proximal end direction with respect to the inner tube 42.

In addition, along with such displacement, as shown in FIG. 3B, the engaging end sections 72 of the arms 46 project toward the distal end from the arm accommodating section 80 and then the arms 46 are displaced (widened) outwardly by the elastic restoring forces thereof. As a result, engagement between the arms 46 provided on the inner tube 42 and the flange 24 provided on the catheter hub 14 is released and, accompanying movement of the inner needle hub 18 in the proximal end direction, the inner tube 42 separates from the catheter hub 14. When this is done, the inner needle 16, which is connected to the inner needle hub 18, also is withdrawn from the catheter 12, whereupon the inner needle unit 21 becomes detached from the catheter member 26. As a result, in the catheter assembly 10, a state is brought about in which only the catheter member 26 is left indwelling on the side of the patient.

In a state in which the inner needle unit 21 has been separated from the catheter member 26, the total length of the inner needle 16 is accommodated in the interior of the protector 20 and the inner needle hub 18, and a condition is brought about in which the tip 17 of the inner needle 16 is covered. On the other hand, after the inner needle 16 has been withdrawn from the catheter 12, a non-illustrated connector of an infusion tube is connected to the proximal end side of the catheter hub 14, whereby an infusion agent (medicinal solution) may be supplied from the infusion tube to the patient.

Incidentally, when the above-described catheter assembly 10 is manufactured, for inserting the inner needle 16 through the insertion hole 55 of the inner tube 42, an assembly method may be considered in which, for example, the proximal end of the inner needle 16 is inserted from the distal end side of the inner tube 42, in a state in which the shutter member 48 is housed in the shutter accommodating section 50, and the inner needle 16 is moved in the proximal end direction relative to the inner tube 42.

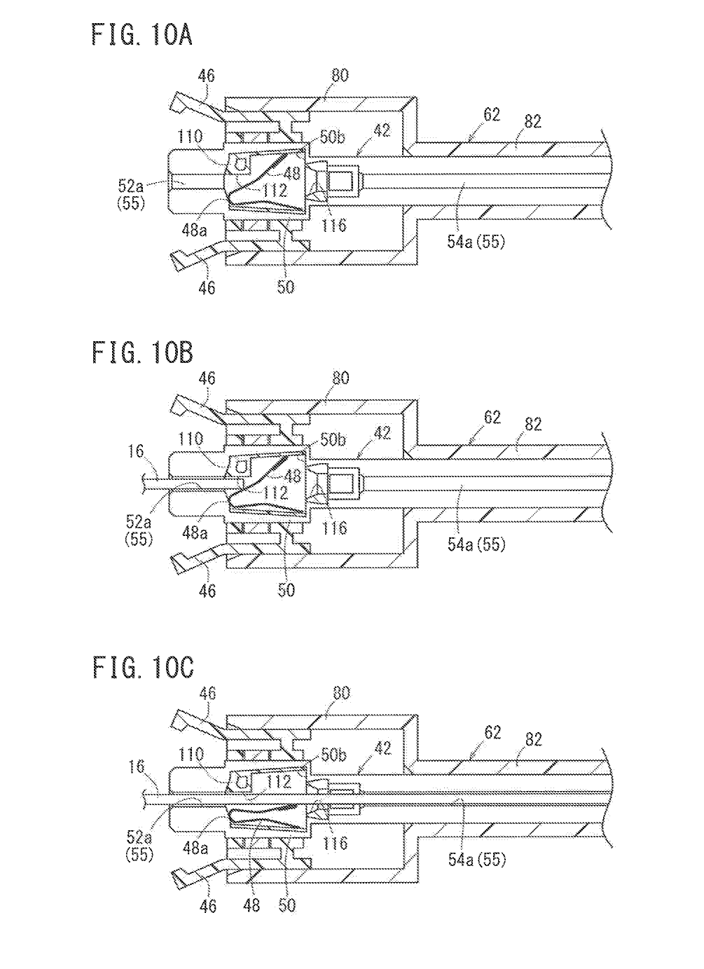

In this case, at a time prior to insertion of the inner needle 16 into the inner tube 42, the V-shaped shutter member 48, which is disposed in the interior of the shutter accommodating section 50 of the inner tube 42, is expanded, as shown in FIG. 10A. Further, due to the needle guide 110 being provided, the distal end portion 48a (V-shaped apex) of the shutter member 48 can easily be located near to one side (a side opposite to the needle guide 110) in the interior of the shutter accommodating section 50. Stated otherwise, in a state in which the shutter member 48 is disposed inside the shutter accommodating section 50, the distal end portion 48a of the shutter member 48 can easily be arranged at a position away from the front of the distal end side lumen 52a.

When the proximal end of the inner needle 16 is inserted through the distal end side lumen 52a from the distal end side of the inner tube 42 in the above state, as shown in FIG. 10B, the proximal end of the inner needle 16 contacts the shutter member 48. In this case, since the distal end portion 48a of the shutter member 48 is at a position distanced from the front of the distal end side lumen 52a, engagement (locking) of the proximal end of the inner needle 16 with the distal end portion 48a of the shutter member 48 can be prevented or suppressed. Accordingly, at an initial stage in which the inner needle 16 is inserted from the distal end side of the inner tube 42, movement of the inner needle 16 relative to the inner tube 42 is not obstructed.

Additionally, as shown in FIG. 10C, when the inner needle 16 is moved further in the proximal end direction relative to the inner tube 42, the shutter member 48 is elastically deformed in a direction to close the V-shape thereof. At this time, although the inner needle 16 receives a reaction force from the shutter member 48, because the inner needle 16 is supported laterally by the needle guide 110, deformation (deflection) of the inner needle 16 as a result of the reaction force from the shutter member 48 is suppressed.

In particular, since the needle guide 110 has the guide surface 112 that continues from the proximal end of the distal end side lumen 52a, the inner needle 16 that projects out from the distal end side lumen 52a is guided effectively by the guide surface 112. Consequently, since the proximal end of the inner needle 16 is prevented from coming into abutment and engaging with (being obstructed by) a proximal end side inner wall 50b of the shutter accommodating section 50, the proximal end of the inner needle 16 can be inserted reliably into the proximal end side lumen 54a. In this case, since the tapered inner surface 116 that expands radially in the distal end direction is provided on the distal end portion of the proximal end side lumen 54a, the proximal end of the inner needle 16 can be introduced suitably into the interior of the proximal end side lumen 54a.

If the proximal end of the inner needle 16 is inserted into the proximal end side lumen 54a, the inner needle 16 is moved further in the proximal end direction relative to the inner tube 42, and the inner needle 16 is inserted into the interior of the inner needle retaining member 36 (see FIGS. 1 and 2) of the inner needle hub 18, which is disposed on the proximal end side of the inner tube 42. The inner needle 16 and the inner needle retaining member 36 are then fixed together mutually, for example, by an adhesive or the like.

According to the catheter assembly 10 of the present embodiment, as described above, because the needle guide 110 is provided in the shutter accommodating section 50, the distal end portion 48a of the shutter member 48 can be easily positioned to one side (a side opposite to the needle guide 110) in the interior of the shutter accommodating section 50. Therefore, during the assembly process, when the inner needle 16 is inserted from the distal end side of the inner tube 42, it is difficult for the proximal end of the inner needle 16 to become caught on the distal end portion 48a of the shutter member 48.

Furthermore, during the assembly process, when the proximal end of the inner needle 16 is made to move in the proximal end direction in the interior of the shutter accommodating section 50 while the shutter member 48 is elastically compressed and deformed, although the inner needle 16 receives a reaction force from the shutter member 48, because the inner needle 16 is supported laterally by the needle guide 110, deformation (deflection) of the inner needle 16 as a result of the reaction force from the shutter member 48 is suppressed. Thus, the proximal end of the inner needle 16 can be inserted smoothly into the proximal end side lumen 54a of the inner tube 42.

Consequently, with the catheter assembly 10, during the assembly process, the inner needle 16 can easily be inserted into the lumen of the inner tube 42 in a state in which the shutter member 48 is accommodated therein.

In the present embodiment, the distal end portion 48a of the shutter member 48 is disposed between the needle guide 110 and the inside wall 51 that faces the needle guide 110 in the shutter accommodating section 50. In accordance with this configuration, the distal end portion 48a of the shutter member 48 can effectively be shifted toward one side of the shutter accommodating section 50 and, during the assembly process, engagement of the proximal end of the inner needle 16 with the distal end portion 48a of the shutter member 48 can be more effectively suppressed.

In the present embodiment, the shutter member 48 is formed in a V-shape, and an apex of the V-shape makes up the distal end portion 48a of the shutter member 48. According to this arrangement, the shutter member 48 that is formed in a V-shape is disposed appropriately and ease of assembly is enhanced.

In the present embodiment, the needle guide 110 has the guide surface 112 that continues from a proximal end of the distal end side lumen 52a. According to this above structure, during the assembly process, upon insertion of the inner needle 16 from the distal end of the inner tube 42, because the inner needle 16, which projects out from the distal end side lumen 52a, is guided effectively by the guide surface 112, the inner needle 16, which receives the reaction force from the shutter member 48, is supported effectively. Thus, the proximal end of the inner needle 16 can be guided more smoothly into the proximal end side lumen 54a.

In the present embodiment, as shown in FIG. 8, the distal end portion of the proximal end side lumen 54a includes the tapered inner surface 116 with an inner diameter that expands outwardly in the direction of the distal end. In addition, the radius r of the distal end opening of the proximal end side lumen 54a is greater than the distance L3 between the guide surface 112 and a common center line a that is shared by the distal end side lumen 52a and the proximal end side lumen 54a. According to this structure, when the inner needle 16 is inserted from the distal end of the inner tube 42, even in the case that the inner needle 16 undergoes a deflection due to the reaction force from the shutter member 48, the tapered inner surface 116 of the proximal end side lumen 54a, which opens widely and outwardly relative to the position of the guide surface 112, allows for the smooth insertion of the proximal end of the inner needle 16 into the proximal end side lumen 54a.

Further, in the present embodiment, as shown in FIG. 8, the distance L1 from the distal end of the distal end side lumen 52a to a proximal end of the guide surface 112 is greater than the distance L2 from the proximal end of the guide surface 112 to the proximal end of the tapered inner surface 116. According to this structure, by setting a portion (the distal end side lumen 52a and the guide surface 112) that supports the inner needle 16 in an elongated manner, deflection of the inner needle 16 by the reaction force received from the shutter member 48 can be suitably suppressed. Accordingly, the proximal end of the inner needle 16 can be inserted more smoothly into the proximal end side lumen 54a.

Furthermore, in the present embodiment, as shown in FIG. 8, the diameter R1 of the proximal end of the distal end side lumen 52a is less than the diameter R2 of a portion of the proximal end side lumen 54a that is disposed more proximally than the tapered inner surface 116. According to this structure, the proximal end of the inner needle 16 can be inserted more smoothly into the proximal end side lumen 54a.

Although preferred embodiments of the present invention has been described, the present invention is not limited to the above-described embodiment. It goes without saying that various modifications can be adopted therein without departing from the scope of the invention.

What is claimed is:

1. A catheter assembly comprising:
an inner needle having a sharp tip at a distal end thereof;
a catheter in which the inner needle is disposed;
a tip protective member having an insertion hole through which the inner needle is insertable, the tip protective member being configured to cover at least a tip of the inner needle when the inner needle is withdrawn; and
a shutter member which is arranged in an interior of a shutter accommodating section provided in the tip protective member, the shutter member being configured to be deformed and thereby block a movement path of the inner needle in the shutter accommodating section when the inner needle is retracted to a predetermined position relative to the tip protective member;
wherein the shutter accommodating section includes a needle guide configured to guide movement of the inner needle.

2. The catheter assembly according to claim 1, wherein a distal end portion of the shutter member is disposed between the needle guide and an inside wall that faces the needle guide in the shutter accommodating section.

3. The catheter assembly according to claim 2, wherein the shutter member is formed in a V-shape, and an apex of the V-shape makes up the distal end portion of the shutter member.

4. The catheter assembly according to claim 1, wherein:
the insertion hole includes an accommodating space in the interior of the shutter accommodating section, a distal end side lumen that extends from a distal end of the accommodating space, and a proximal end side lumen that extends from a proximal end of the accommodating space; and
the needle guide includes a guide surface that continues from a proximal end of the distal end side lumen.

5. The catheter assembly according to claim 4, wherein:
a distal end portion of the proximal end side lumen includes a tapered inner surface with an inner diameter that expands outwardly in a direction of the distal end; and
a radius of a distal end opening in the tapered inner surface of the proximal end side lumen is greater than a distance between the guide surface and a common center line shared by the distal end side lumen and the proximal end side lumen.

6. The catheter assembly according to claim 4, wherein:
a distal end portion of the proximal end side lumen includes a tapered inner surface with an inner diameter that expands outwardly in a direction of the distal end; and
a distance from a distal end of the distal end side lumen to a proximal end of the guide surface is greater than a distance from the proximal end of the guide surface to a proximal end of the tapered inner surface.

7. The catheter assembly according to claim 4, wherein:
a distal end portion of the proximal end side lumen includes a tapered inner surface with an inner diameter that expands outwardly in a direction of the distal end; and
a diameter of the proximal end of the distal end side lumen is less than a diameter of a portion of the proximal end side lumen that is disposed more proximally than the tapered inner surface.
